(19) United States
(12) Patent Application Publication
(10) Pub. No.: US 2023/0255683 A1
(43) Pub. Date: Aug. 17, 2023

(54) ELONGATED MEDICAL NEEDLE ASSEMBLY

(71) Applicant: Boston Scientific Medical Device Limited, Galway (IE)

(72) Inventor: Isha Warikoo, Oakville (CA)

(21) Appl. No.: 18/304,086

(22) Filed: Apr. 20, 2023

Related U.S. Application Data
(63) Continuation of application No. PCT/IB2021/059631, filed on Oct. 19, 2021.
(60) Provisional application No. 63/093,929, filed on Oct. 20, 2020.

(51) Int. Cl. A61B 18/14

(52) U.S. Cl. CPC A61B 18/1477; A61B 18/1492; A61B 2018/00077

(57) ABSTRACT

An elongated medical needle assembly includes an elongated electrically-conductive flexible tube assembly configured to be maneuvered, at least in part, within the patient and toward the biological feature. An electrically-exposed outer surface is located between spaced-apart electrically-insulated layers, and covers, at least in part, an outer surface of the elongated electrically-conductive flexible tube assembly. The electrically-exposed outer surface is configured to selectively emit energy toward the biological feature of the patient.

### CROSS-REFERENCE TO RELATED APPLICATIONS

This application is a continuation of and claims the benefit of International Application Number PCT/IB2021/059631, entitled “ELONGATED MEDICAL NEEDLE ASSEMBLY,” and filed Oct. 19, 2021, which claims the benefit of U.S. Provisional Application Number 63/093,929, entitled “ELONGATED MEDICAL NEEDLE ASSEMBLY,” and filed Oct. 20, 2020, which are hereby incorporated by reference in their entireties.

### TECHNICAL FIELD

This document relates to the technical field of (and is not limited to) an elongated medical needle assembly (and method therefor).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with existing medical needles (also called the existing technology). After much study of, and experimentation with, the existing medical needles, an understanding (at least in part) of the problem and its solution have been identified (at least in part) and are articulated (at least in part) as follows:

In view of the known systems, what may be needed is an apparatus for selectively emitting energy toward a biological feature of a patient from an elongated medical needle assembly.

To mitigate, at least in part, at least one problem associated with the existing technology, there is provided (in accordance with a major aspect) an apparatus. The apparatus is for use on (with) a biological feature of a patient. The apparatus includes and is not limited to (comprises) an elongated medical needle assembly including an elongated electrically-conductive flexible tube assembly configured to be maneuvered, at least in part, within the patient and toward the biological feature. An electrically-exposed outer surface is located between spaced-apart electrically-insulated layers, and covers, at least in part, an outer surface of the elongated electrically-conductive flexible tube assembly. It will be appreciated that the hatch marking in all sketches (as for example in FIG. 1) represent an insulative coating (such as, the spaced-apart electrically-insulated layers. The electrically-exposed outer surface is configured to selectively emit energy toward the biological feature of the patient.

In view of the known systems, what may be needed is an apparatus for selectively emitting energy toward a biological feature of a patient without emitting energy from a distal portion of an elongated medical needle assembly.

To mitigate, at least in part, at least one problem associated with the existing technology, there is provided (in accordance with a major aspect) an apparatus for selectively emitting energy toward a biological feature of a patient without emitting energy from a distal portion of an elongated medical needle assembly.

In view of the known systems, what may be needed is an apparatus for selectively emitting energy toward a biological feature of a patient without emitting energy from a distal portion positioned at an exit portal of an elongated lumen defined by an elongated medical needle assembly.

To mitigate, at least in part, at least one problem associated with the existing technology, there is provided (in accordance with a major aspect) an apparatus for selectively emitting energy toward a biological feature of a patient without emitting energy from a distal portion positioned at an exit portal of an elongated lumen defined by an elongated medical needle assembly.

To mitigate, at least in part, at least one problem associated with the existing technology, there is provided (in accordance with a major aspect) a method. The method is for using an elongated medical needle assembly including an elongated electrically-conductive flexible tube assembly. The method includes and is not limited to (comprises) maneuvering the elongated electrically-conductive flexible tube assembly into a patient and toward a biological feature of the patient. The elongated electrically-conductive flexible tube assembly has a distal portion. A first electrically-insulated layer covers, at least in part, an outer surface of the elongated electrically-conductive flexible tube assembly. A second electrically-insulated layer covers, at least in part, the distal portion of the elongated electrically-conductive flexible tube assembly. An electrically-exposed outer surface is located proximate to the distal portion between the first electrically-insulated layer and the second electrically-insulated layer. The electrically-exposed outer surface is configured to selectively emit energy toward the biological feature of the patient in response to selective movement of the energy along the elongated electrically-conductive flexible tube assembly toward the electrically-exposed outer surface. The method also includes selectively emitting energy from the electrically-exposed outer surface toward the biological feature of the patient in response to selective movement of the energy along the elongated electrically-conductive flexible tube assembly toward the electrically-exposed outer surface.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 depicts a side view in accordance with a first embodiment (implementation) of an elongated medical needle assembly; and

FIG. 2 depicts a side view in accordance with a second embodiment (implementation) of an elongated medical needle assembly; and

FIG. 3 and FIG. 4 depict side views in accordance with the first embodiment (implementation) of the elongated medical needle assembly of FIG. 1; and

FIG. 5 and FIG. 6 depict side views in accordance with the second embodiment (implementation) of the elongated medical needle assembly of FIG. 2; and

FIG. 7 and FIG. 8 depict a side view (FIG. 7) and a side perspective view (FIG. 8) of an embodiment of a technical feature (an option) that may be included with any one of (a) the first embodiment of elongated medical needle assembly of FIG. 15, (b) the second embodiment of the elongated medical needle assembly of FIG. 17, and/or (c) a third embodiment of the elongated medical needle assembly of FIG. 19; and

FIG. 9, FIG. 10, FIG. 11 depict side views in accordance with the third embodiment (implementation) of an elongated medical needle assembly; and

FIG. 12A, FIG. 12B, FIG. 13 and FIG. 14 depict side views of embodiments of the technical feature(s) of FIG. 7 and FIG. 8; and

FIG. 15 and FIG. 16 depict side views in accordance with the first embodiment (implementation) of the elongated medical needle assembly of FIG. 1 in combination with the technical feature(s) of FIG. 12A; and

FIG. 17 and FIG. 18 depict side views in accordance with the second embodiment (implementation) of the elongated medical needle assembly of FIG. 2 in combination with the technical feature(s) of FIG. 12A; and

FIG. 19 and FIG. 20 depict side views in accordance with the third embodiment (implementation) of the elongated medical needle assembly of FIG. 9 in combination with the technical feature(s) of FIG. 12A; and

FIG. 21, FIG. 22 and FIG. 23 depict, for the purpose of a side-by-side comparison, a side view (FIG. 21) of the first embodiment (implementation) of the elongated medical needle assembly of FIG. 1, a side view (FIG. 22) of the second embodiment (implementation) of the elongated medical needle assembly of FIG. 2, and a side view (FIG. 23) of the third embodiment (implementation) of the elongated medical needle assembly of FIG. 9; and

FIG. 24 depicts a side view of any embodiment (implementation) of the elongated medical needle assembly of FIG. 1, FIG. 2 or FIG. 9.

The drawings are not necessarily to scale and may be illustrated by phantom lines, diagrammatic representations, and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

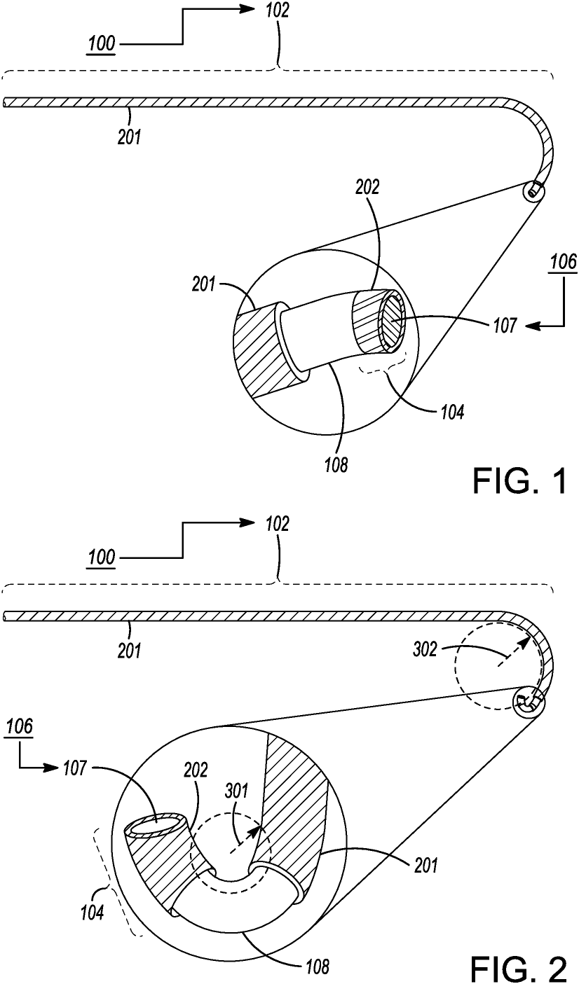

FIG. 1 depicts a side view in accordance with a first embodiment (implementation) of an elongated medical needle assembly 100.

FIG. 2 depicts a side view in accordance with a second embodiment (implementation) of an elongated medical needle assembly 100.

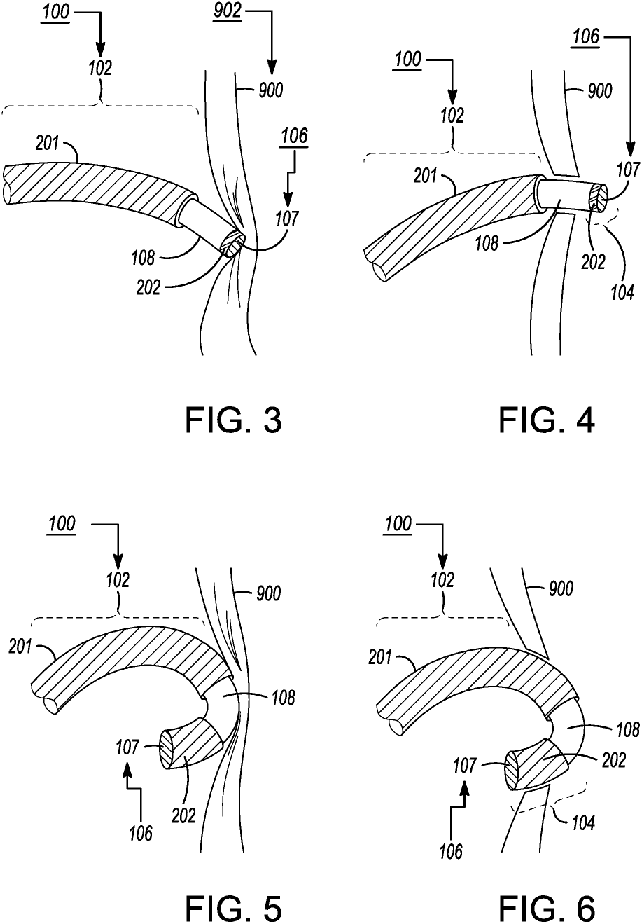

FIG. 3 and FIG. 4 depict side views in accordance with the first embodiment (implementation) of the elongated medical needle assembly 100 of FIG. 1.

FIG. 5 and FIG. 6 depict side views in accordance with the second embodiment (implementation) of the elongated medical needle assembly 100 of FIG. 2.

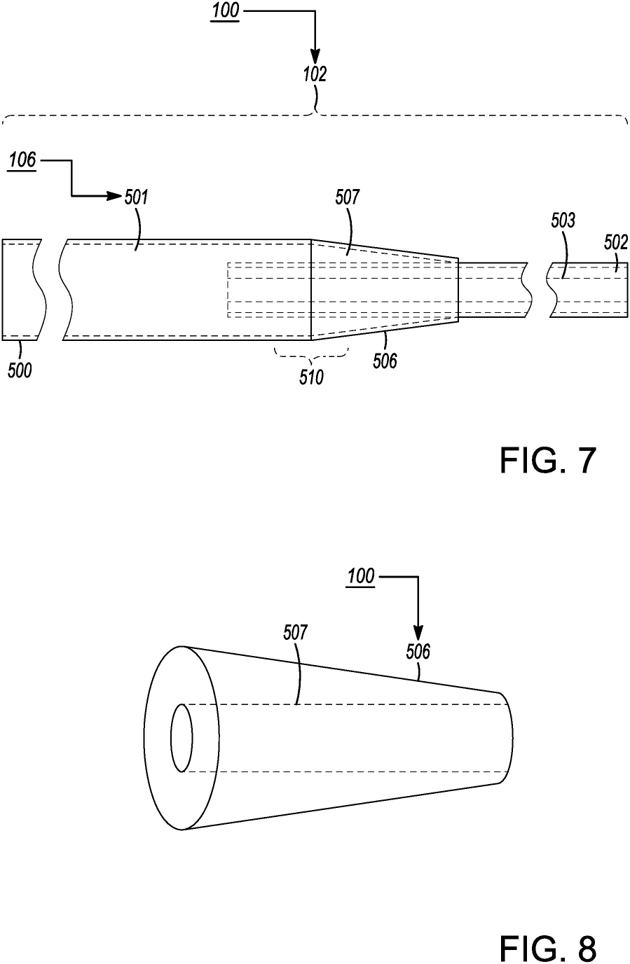

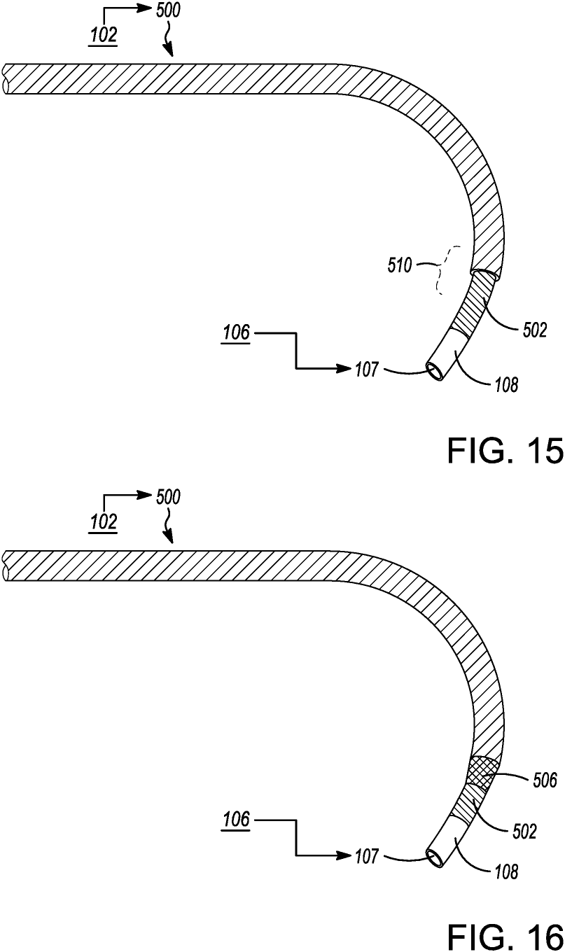

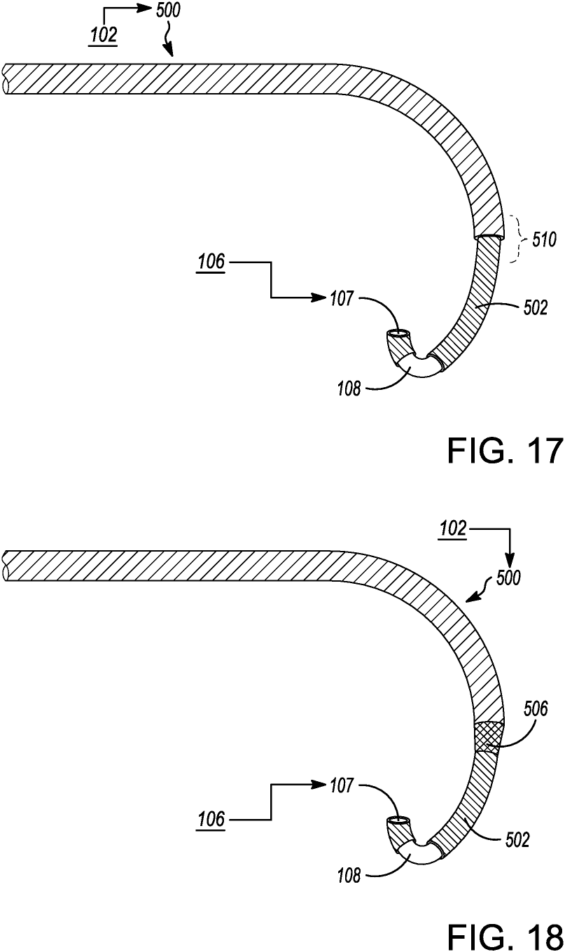

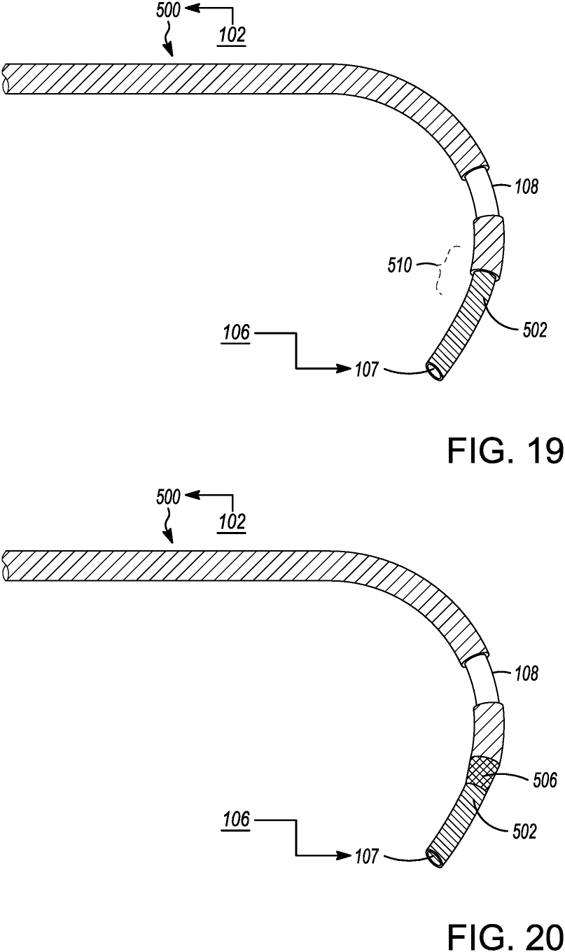

FIG. 7 and FIG. 8 depict a side view (FIG. 7) and a side perspective view (FIG. 8) of an embodiment of a technical feature (an option) that may be included with any one of (a) the first embodiment of elongated medical needle assembly 100 of FIG. 15, (b) the second embodiment of the elongated medical needle assembly 100 of FIG. 17, and/or (c) a third embodiment of the elongated medical needle assembly 100 of FIG. 19.

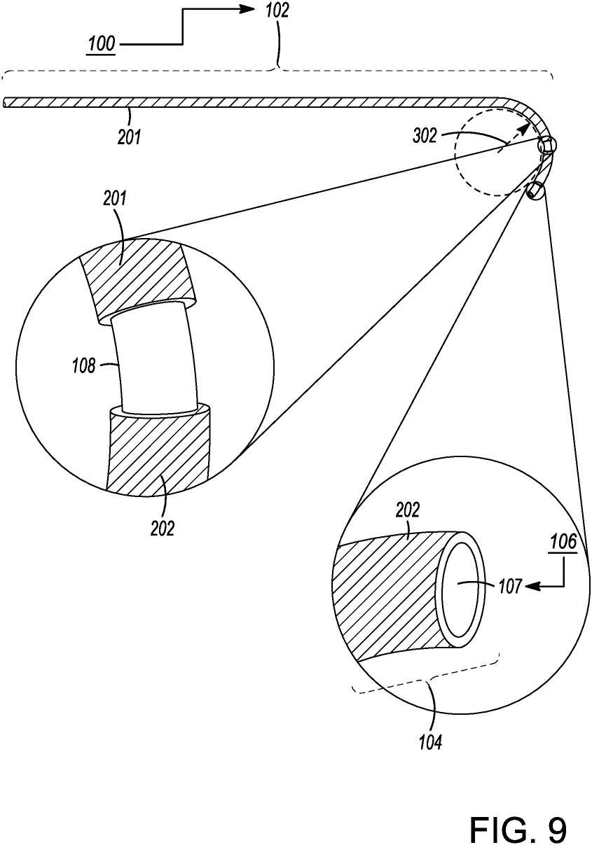

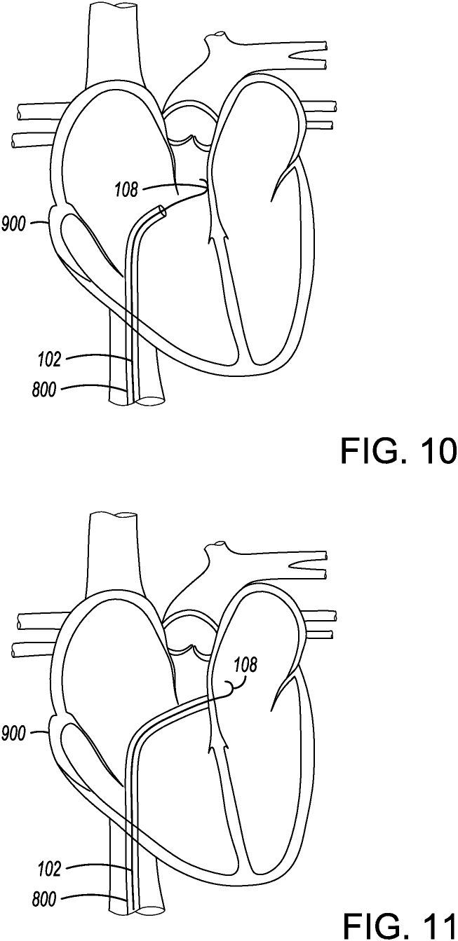

FIG. 9, FIG. 10, FIG. 11 depict side views in accordance with the third embodiment (implementation) of an elongated medical needle assembly 100.

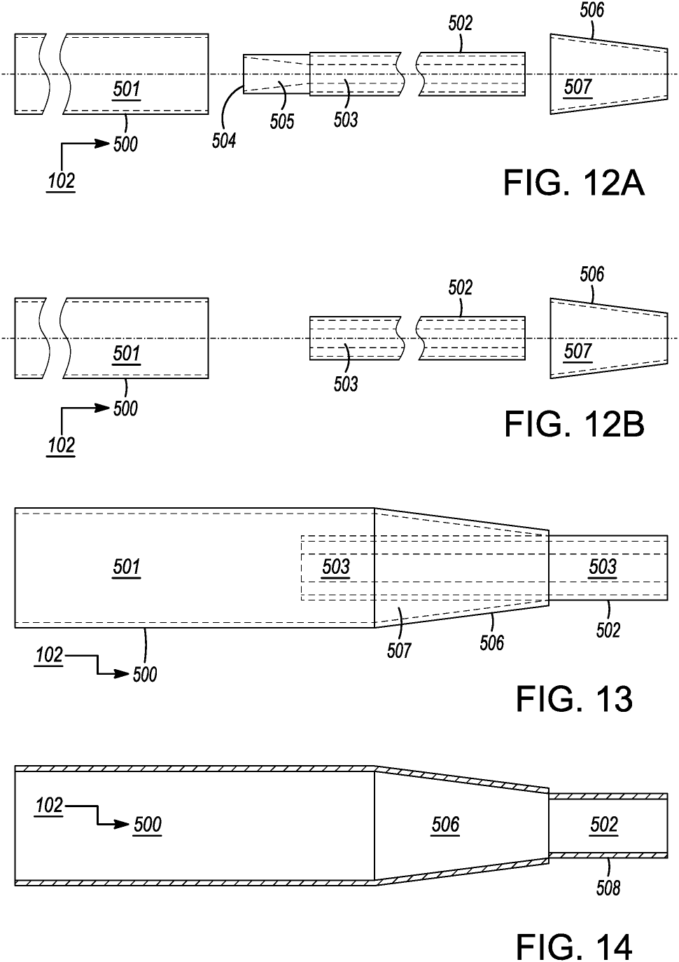

FIG. 12A, FIG. 12B, FIG. 13 and FIG. 14 depict side views of embodiments of the technical feature of FIG. 7 and FIG. 8.

Referring to the embodiment (implementation) as depicted in FIG. 1, the elongated medical needle assembly 100 is configured to be inserted into a confined space defined by a living body of a patient 902, as depicted in FIG. 3 to FIG. 4. The elongated medical needle assembly 100 includes (preferably) a relatively thin and flexible wire or flexible tube (an elongated flexible shaft) configured to be inserted into a confined or tortuous space (a confined space) defined by the living body.

Referring to the embodiment (implementation) as depicted in FIG. 1, the elongated medical needle assembly 100 includes biocompatible materials suitable for specific performance (such as, dielectric strength, thermal, electrical insulation, corrosion, water resistance, heat resistance, etc.), for compliance with industrial and/or regulatory safety standards (or compatible for medical usage), etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

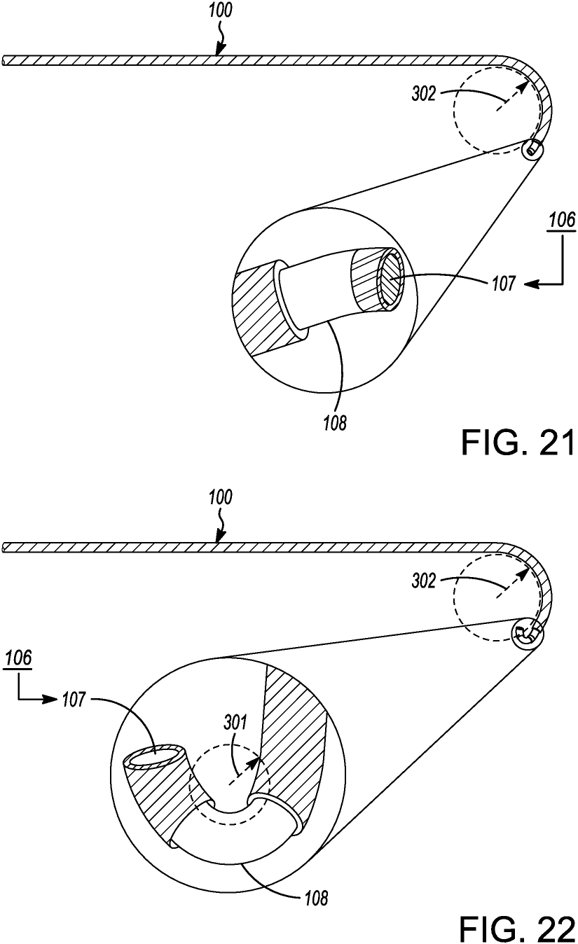

Referring to the embodiment (implementation) as depicted in FIG. 1, the elongated medical needle assembly 100 includes an elongated electrically-conductive flexible tube assembly 102 having a distal portion 104 defining an elongated lumen 106 longitudinally extending along the elongated electrically-conductive flexible tube assembly 102 toward the distal portion 104. The elongated lumen 106 terminates at a lumen portal 107 (located at the distal portion 104). The electrically-exposed outer surface 108 is located adjacent to the distal portion 104 and is located in a spaced-apart relationship to the lumen portal 107. For instance, the elongated electrically-conductive flexible tube assembly 102 may include a shape-memory material configured to be manipulated and/or deformed followed by a return to the original shape that the shape-memory material was set in (prior to manipulation). Shape-memory materials (SMMs) are known and not further described in detail. Shape-memory materials are configured to recover their original shape from a significant and seemingly plastic deformation in response to a particular stimulus being applied to the shape-memory material. This is known as the shape memory effect (SME). Superelasticity (in alloys) may be observed once the shape-memory material is deformed under the presence (an application) of a stimulus force. It will be appreciated that the elongated electrically-conductive flexible tube assembly 102 may include (a) a single tube, (b) a single tapered tube, (c) a proximal and a distal tube positioned one after the other, (d) a proximal and a distal tube positioned one after the other with the outer diameters being different from each other, etc. The elongated electrically-conductive flexible tube assembly 102 are, preferably, configured to meet dimensional and/or geometrical constraints that may allow the elongated medical needle assembly 100 (or the elongated electrically-conductive flexible tube assembly 102) to be inserted into other medical devices, such as a sheath assembly (known and not depicted), a dilator assembly (known and not depicted), etc., and/or or any equivalent. It will be appreciated that the elongated electrically-conductive flexible tube assembly 102 may include a single tube or a single tapered tube as depicted in FIG. 1, FIG. 2 and FIG. 9. It will be appreciated that the elongated electrically-conductive flexible tube assembly 102 may include a two-piece tube assembly (composed of a proximal tube and a distal tube) as depicted in FIG. 15 to FIG. 20. For clarity, it will be appreciated that the first embodiment and options thereof are depicted in FIG. 1, FIG. 3, FIG. 4, FIG. 15, FIG. 16 and/or FIG. 21. For clarity, it will be appreciated that the second embodiment and options thereof are depicted in FIG. 2, FIG. 5, FIG. 6, FIG. 17, FIG. 18 and/or FIG. 22. For clarity, it will be appreciated that the third embodiment and options thereof are depicted in FIG. 9, FIG. 10, FIG. 11, FIG. 19, FIG. 20 and FIG. 23. It will be appreciated that the catheter assembly 800 (as depicted in FIG. 10 and FIG. 11) may include a sheath and dilator assembly and any equivalent thereof.

Referring to the embodiment of a technical feature (an option) as depicted in FIG. 7, that may be included with any one of (a) the first embodiment of elongated medical needle assembly 100 of FIG. 15, (b) the second embodiment of the elongated medical needle assembly 100 of FIG. 17, and/or (c) a third embodiment of the elongated medical needle assembly 100 of FIG. 19 where the medical needle assembly includes two shafts, a proximal and distal shaft. A tapered socket (that is the transition tube 506) is configured to strengthen and/or reduce stress concentration that might develop at the shoulder section 510. The shoulder section 510 may be called a proximal-distal joint, a step, an edge, etc. The shoulder section 510 is also depicted in FIG. 15, FIG. 17 and FIG. 19 without the transition tube 506 installed to the elongated electrically-conductive flexible tube assembly 102 (as an option, if desired). The socket (that is, the transition tube 506) may be shrink-fit to the distal shaft 502. Alternatively, the transition tube 506 may be fixed to the distal shaft 502 at the shoulder section 510, such as, preferably, with glue (an adhesive), such as the EPO-TEK (TRADEMARK) Model number 353 ND epoxy (manufactured by EPOXY TECHNOLOGY, INC., based out of the U.S.A.), and/or any equivalent thereof.

Referring to the embodiment (implementation) as depicted in FIG. 7, the elongated medical needle assembly 100 also includes a first electrically-insulated layer 201 covering, at least in part, an outer surface of the elongated electrically-conductive flexible tube assembly 102. The elongated medical needle assembly 100 also includes a second electrically-insulated layer 202 covering, at least in part, the distal portion 104 of the elongated electrically-conductive flexible tube assembly 102. The elongated electrically-conductive flexible tube assembly 102 includes a proximal tube 500 and a distal tube 502 (further details are associated with FIG. 12A and FIG. 12B).

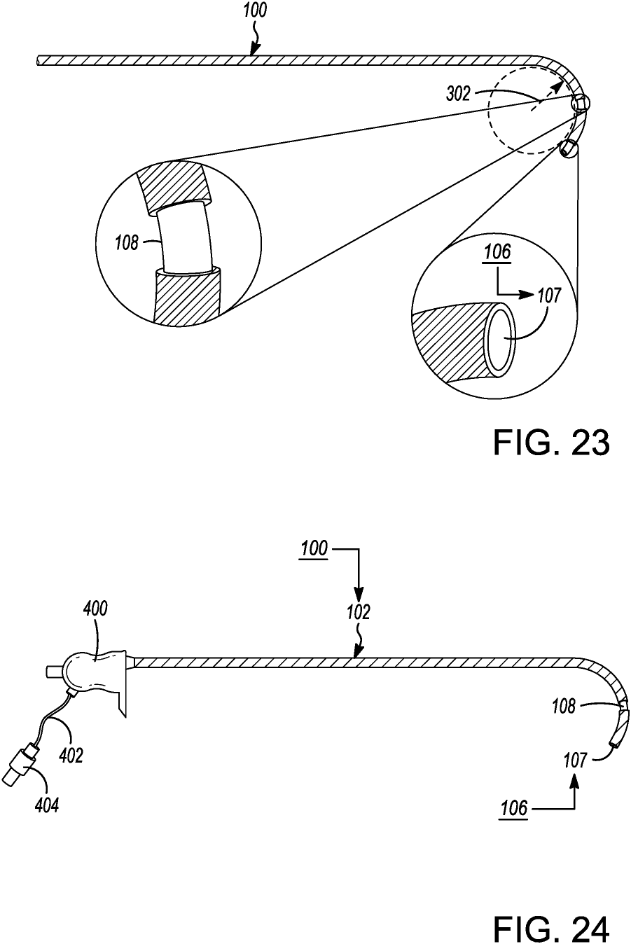

Referring to the embodiment (implementation) as depicted in FIG. 1, the electrically-exposed outer surface 108 is configured to selectively emit energy, preferably similar to a radio frequency puncture device, such as the BAYLIS (TRADEMARK) POWERWIRE (REGISTERED TRADEMARK) radio frequency guidewire manufactured by BAYLIS MEDICAL COMPANY (headquartered in Canada). Referring to the embodiment (implementation) as depicted in FIG. 1, the first electrically-insulated layer 201 and second electrically-insulated layer 202 includes a coating of a Polytetrafluoroethylene (PTFE) heat-shrink insulation, a paralene dielectric coating, and/or any equivalent thereof. The electrically-exposed outer surface 108 (also called an active region) is configured to selectively emit energy for puncturing tissue. The electrically-exposed outer surface 108 may act as an electrode. The electrically-exposed outer surface 108 is configured to selectively emit energy (such as radiofrequency energy). The electrically-exposed outer surface 108 is characterized as a region (small region) of exposed metal located between the first electrically-insulated layer 201 and the second electrically-insulated layer 202. In accordance with a preferred embodiment, the elongated lumen extends throughout the entire length of the elongated electrically-conductive flexible tube assembly 102. In accordance with an optional embodiment, reference is made to FIG. 7 and FIG. 12A in which the elongated lumen extends throughout the entire length of the elongated electrically-conductive flexible tube assembly 102 (that is, the elongated lumen extends between the proximal tube 500 and the distal tube 502). Preferably, the lumen extends throughout the entire needle assembly, and through the handle assembly 400 (as depicted in in FIG. 24). It is preferred to have the distal portion 104 not emit energy (such as radiofrequency energy). The electrically-exposed outer surface 108 is a region of exposed metal that interrupts the first electrically-insulated layer 201 and the second electrically-insulated layer 202. For the case where it may be desired to avoid tissue coring, the elongated lumen 106 terminating at the distal portion 104 is coated or insulated (with an electrically insulative material) to ensure there is no risk of inadvertent tissue coring of a biological feature (thereby avoiding formation of a free-floating particle that might cause an embolism, etc.). The electrically-exposed outer surface 108, preferably, forms or includes a surface roughness that is greater than the surface roughness of the elongated electrically-conductive flexible tube, and/or first electrically-insulated layer 201 and/or the second electrically-insulated layer 202 (as the increased friction between the interacting surfaces may stabilize contact between the electrically-exposed outer surface 108 and a desired puncture site located on the biological feature to be punctured by the electrically-exposed outer surface 108). The location of the electrically-exposed outer surface 108 may allow for an atraumatic, unoccluded open lumen at the distal portion 104 (if desired). The elongated electrically-conductive flexible tube assembly 102 may be used in minimally invasive cardiac procedures, allowing surgeons to gain transseptal access by puncturing the fossa ovalis in the heart, etc. The elongated electrically-conductive flexible tube assembly 102 may be applicable to similar areas of use, provided that the constraints and requirements of the procedure are similar to that of a minimally invasive transseptal access cardiac surgery.

Referring to the embodiment (implementation) as depicted in FIG. 1, the elongated electrically-conductive flexible tube assembly 102 may include a single tapered tube (or hypotube), etc. The tapered geometry of the elongated electrically-conductive flexible tube assembly 102 may, if desired, ensure that the elongated medical needle assembly 100 is compatible with an accessory device (such as a sheath and/or a dilator, etc.).

Referring to the embodiment (implementation) as depicted in FIG. 1, the elongated electrically-conductive flexible tube 102 includes SAE (Society of Automotive Engineering) Type 304 stainless steel (suitable, for instance, with transseptal access puncture devices). Additionally, Type 304 stainless steel is biocompatible, conductive and possesses suitable material properties (such as, stiffness) for a given application and/or procedure, etc.

Type 304 stainless steel contains both chromium (from between about 15% to about 20%) and nickel (from between about 2% to about 10.5%) metals as the main non-iron constituents.

Referring to the embodiment (implementation) as depicted in FIG. 1, the electrically-exposed outer surface 108 is located at the distal-most end of the elongated electrically-conductive flexible tube 102 when the elongated electrically-conductive flexible tube 102 is in its original, curved shape. The electrically-exposed outer surface 108 (a region of exposed metal) is configured to act as an electrode, which administers energy (radiofrequency energy) to puncture the tissue. The electrically-exposed outer surface 108 may be composed of Type 304 stainless steel plated with platinum. The use of platinum ensures the electrically-exposed outer surface 108 is radiopaque and may be visualized under fluoroscopy and/or echocardiography. For instance, the electrically-exposed outer surface 108 may require a width of at least about 0.03 inches. The electrically-exposed outer surface 108 may be large enough to puncture tissue by emitting energy; however, small enough to ensure the puncture hole heals post-surgery. The electrically-exposed outer surface 108 may be created by heat-shrinking two separate lengths of electrically insulative sections to the elongated electrically-conductive flexible tube 102 before or after curving of the elongated electrically-conductive flexible tube 102. Alternatively, the electrical insulation may be applied to the entire length of the elongated electrically-conductive flexible tube 102, and a section of the insulation may be cut away (by using a razor blade or equivalent method), to expose the electrically-exposed outer surface 108. Hot-air dipping may be used to seal the insulation along the distal portion 104.

Referring to the embodiment (implementation) as depicted in FIG. 1, the first electrically-insulated layer 201 and the second electrically-insulated layer 202 (electrical insulation) cover, preferably, the entire length of the elongated electrically-conductive flexible tube 102 excluding the electrically-exposed outer surface 108. The first electrically-insulated layer 201 and the second electrically-insulated layer 202 are highly lubricious, allowing easy movement (advancement and/or retraction) of the elongated electrically-conductive flexible tube 102 from an accessory device and/or patient vasculature. Any equivalent insulative material that meets all mechanical performance, electrically insulative properties and biocompatibility requirements may be used.

Referring to the embodiment (implementation) as depicted in FIG. 24, a molded plastic handle with a curve indicator (known and not depicted) may be installed to the elongated medical needle assembly 100. The handle allows surgeons to navigate through patient anatomy and guide the distal portion 104 with greater ease. The curve indicator points to the direction in which the elongated medical needle assembly 100 is curving, which allows the user to maneuver the device accordingly. The handle merely improves ease of use.

Referring to the embodiment (implementation) as depicted in FIG. 24, any method that facilitates an electrical connection to pass energy (radiofrequency energy) to the electrically-exposed outer surface 108 may be sufficient. The length of the elongated medical needle assembly 100 may be any suitable length for a given procedure such as for reaching the fossa ovalis from surgical entry in the upper thigh.

Referring to the embodiment (implementation) as depicted in FIG. 24, the electrically-exposed outer surface 108 is, preferably, configured to be usable with any type of energy generator, such as the BAYLIS MEDICAL MODEL RPA-100A energy generator (or equivalent) configured to transmit (generate) radiofrequency energy. A cable (known and not depicted) supports electrical connection between the electrically-exposed outer surface 108 and the generator.

Referring to the embodiment (implementation) as depicted in FIG. 2, the first electrically-insulated layer 201 covers the entirety of the elongated electrically-conductive flexible tube 102 (except for the electrically-exposed outer surface 108). The electrically-exposed outer surface 108 may be characterized as exposed metal at the top of the J-curve of the elongated electrically-conductive flexible tube 102. Preferably, a length (a section of the shaft) of the elongated electrically-conductive flexible tube assembly 102 forms two curves at two spaced-apart sections thereof: a first curve having a first radius 301 located at the distal portion 104, and a second curve having a second radius 302 (a relatively larger curve) (that is spaced apart from the distal portion 104). The electrically-exposed outer surface 108 (the electrode) is located at the distal-most end of the smaller curve, when in its original, curved shape. The electrically-exposed outer surface 108 is located proximate to the distal portion 104 and elongated lumen 106 (that is, proximate to the open lumen face).

FIG. 3 to FIG. 4 depict side views of embodiments (implementations) of the elongated medical needle assembly 100 of FIG. 1.

Referring to the embodiment (implementation) as depicted in FIG. 3, the elongated electrically-conductive flexible tube assembly 102 is configured to be maneuvered, at least in part, within the patient 902 and toward the biological feature 900. The electrically-exposed outer surface 108 is located between spaced-apart electrically-insulated layers (201, 202) covering, at least in part, an outer surface of the elongated electrically-conductive flexible tube assembly 102. The electrically-exposed outer surface 108 is configured to selectively emit radiofrequency energy toward the biological feature 900 of the patient 902.

Referring to the embodiment (implementation) as depicted in FIG. 3, the elongated electrically-conductive flexible tube assembly 102 has a distal portion 104 configured to be maneuvered, at least in part, within the patient 902 and toward the biological feature 900. The first electrically-insulated layer 201 covers, at least in part, an outer surface of the elongated electrically-conductive flexible tube assembly 102. The second electrically-insulated layer 202 covers, at least in part, the distal portion 104 of the elongated electrically-conductive flexible tube assembly 102. The electrically-exposed outer surface 108 is located proximate to the distal portion 104. The electrically-exposed outer surface 108 is also located between the first electrically-insulated layer 201 and the second electrically-insulated layer 202. The electrically-exposed outer surface 108 is configured to selectively emit energy toward the biological feature 900 of the patient 902 in response to selective movement of the energy along the elongated electrically-conductive flexible tube assembly 102 toward the electrically-exposed outer surface 108.

Referring to the embodiments (implementations) as depicted in FIG. 3 to FIG. 6, there is depicted a method of using an elongated medical needle assembly 100. The method may be applicable to all of the embodiments of the elongated electrically-conductive flexible tube assembly 102. The method includes maneuvering the elongated electrically-conductive flexible tube assembly 102 into the patient 902 and toward the biological feature 900 of the patient 902 (as depicted in FIG. 3 or FIG. 5). The method also includes selectively emitting energy from the electrically-exposed outer surface 108 toward the biological feature 900 of the patient 902 in response to selective movement of the energy along the elongated electrically-conductive flexible tube assembly 102 toward the electrically-exposed outer surface 108 (as depicted in FIG. 3 or FIG. 5). In this manner, the electrically-exposed outer surface 108 may then form a puncture hole through the biological feature 900 of the patient 902 (as depicted in FIG. 4 and FIG. 6).

Referring to the embodiments (implementations) as depicted in FIG. 4 and FIG. 6 the electrically-exposed outer surface 108 (electrode) is located at the distal portion 104 of the elongated electrically-conductive flexible tube 102, thereby allowing a user to advance the elongated electrically-conductive flexible tube 102 when the biological feature 900 (such as the septum) is initially punctured. The electrically-exposed outer surface 108 is coated in an electrically-insulative material to prevent tissue coring and/or risk of causing an embolism.

Referring to the embodiments (implementations) as depicted in FIG. 5 and FIG. 6, the elongated electrically-conductive flexible tube 102 includes a J-curve extending from the distal portion 104. The J-curve is configured, preferably, to advance through the septum when the tissue is initially punctured.

Referring to the embodiment (implementation) as depicted in FIG. 5 and FIG. 6 (which may be applicable to the embodiment as depicted in FIG. 3 and FIG. 4) (that is, to depict the workflow), the method (workflow) may include: (a) advancing the elongated electrically-conductive flexible tube assembly 102 toward the biological feature 900 of the patient 902 (that is, into patient vasculature); and (b) using the electrically-exposed outer surface 108 to tent the biological feature 900 (such as the septum or the heart); and (c) activating the emission of energy (radiofrequency) so that the electrically-exposed outer surface 108, in use, emits energy toward the biological feature 900 that is being tented (so that the biological feature 900 may become punctured); and (d) maneuvering the elongated electrically-conductive flexible tube assembly 102 to cross the biological feature 900, leading with the electrically-exposed outer surface 108 (electrode-first), immediately once (after) the puncture is made (formed; the curve at the distal tip is small enough to pass through the puncture hole.

FIG. 7 and FIG. 8 depict a side view (FIG. 7) and a side perspective view (FIG. 8) of an embodiment of a technical feature (an option) that may be included with any one of (a) the first embodiment of elongated medical needle assembly 100 of FIG. 15, (b) the second embodiment of the elongated medical needle assembly 100 of FIG. 17, and/or (c) a third embodiment of the elongated medical needle assembly 100 of FIG. 19.

Referring to the embodiment (implementation) as depicted in FIG. 8, the elongated electrically-conductive flexible tube assembly 102 may include two (2) spaced-apart tubes (or hypotubes) such as a larger diameter proximal shaft section and a smaller diameter distal shaft section, with the electrically-exposed outer surface 108 positioned therebetween. In order to reduce the risk of bending/breaking at the proximal-distal joint 510, a tapered socket may be included at the joint. The socket may be glued or shrink-fit to the joint, prior to application of electrical insulation. The taper length of the socket may be enough to minimize the risk of fracture at the proximal-distal joint and along the socket boundaries. Referring to the embodiment (implementation) as depicted in FIG. 8, the socket may be composed of SAE (Society of Automotive Engineering) Type 304 stainless steel (or equivalent). Additionally, Type 304 stainless steel is biocompatible, conductive and possesses suitable material properties (such as, stiffness) for a given application and/or procedure, etc.

FIG. 10 and FIG. 11 (SHEET 4 of 4 sheets) depict side views of embodiments (implementations) of the elongated medical needle assembly 100 of FIG. 9.

FIG. 10 and FIG. 11 (SHEET 4 of 4 sheets) depict side views of embodiments (implementations) of the elongated medical needle assembly 100 of FIG. 9.

Referring to the embodiment (implementation) as depicted in FIG. 9 (which is an alternative to the embodiment as depicted in FIG. 1, and FIG. 2), the shape of the elongated electrically-conductive flexible tube assembly 102 (as depicted FIG. 9) is not identical to the embodiments as depicted in FIG. 1, and FIG. 2). The length (a section of the shaft) of the elongated electrically-conductive flexible tube assembly 102 forms a single curve having a second radius 302 (that is, a single radius).

Referring to the embodiment (implementation) as depicted in FIG. 9, the electrically-exposed outer surface 108 (the electrode) is located at a distal-most section of the single curve (when in the elongated electrically-conductive flexible tube assembly 102 is in its original, curved shape, as depicted in FIG. 9). The electrically-exposed outer surface 108 is spaced apart from the entrance (the portal lumen 107 or the open lumen face) of the elongated lumen 106 at the distal portion 104. The electrically-exposed outer surface 108 may (if desired) have a surface roughness greater than the proximal shaft of the elongated electrically-conductive flexible tube assembly 102 (to improve contact with the biological feature 900 (such as the septum of the heart) when tenting the distal portion 104 against the biological feature 900, etc.

Referring to the embodiment (implementation) as depicted in FIG. 10 and FIG. 11 (to depict the workflow), the method (workflow) may include: (a) advancing the elongated electrically-conductive flexible tube assembly 102 toward the biological feature 900 of the patient 902 (that is, into patient vasculature); and (b) using the electrically-exposed outer surface 108 to tent the biological feature 900 (such as the septum or the heart); and (c) activating the emission of energy (radiofrequency) so that the electrically-exposed outer surface 108 emits energy toward the biological feature 900 that is being tented (so that the biological feature 900 may become punctured); and (d) maneuvering the elongated electrically-conductive flexible tube assembly 102 to ensure that the biological feature 900 is crossed distal tip-first (the distal curve 302, is too large to directly pass through the puncture hole that was formed through the biological feature 900).

FIG. 12A, FIG. 12B, FIG. 13 and FIG. 14 depict side views of embodiments of the technical feature(s) of FIG. 7 and FIG. 8.

Referring to the embodiment (implementation) as depicted in FIG. 12A, the elongated electrically-conductive flexible tube assembly 102 includes a proximal tube 500 and a distal tube 502. The outer diameter of the proximal tube 500 is larger than the outer diameter of the distal tube 502. The proximal tube 500 defines a proximal lumen 501 extending along a longitudinal axis of the proximal tube 500. The distal tube 502 defines a distal lumen 503 extending along a longitudinal axis of the distal tube 502. A portion of the distal tube 502 is received (at least in part) into the proximal lumen 501 of the proximal tube 500. For instance, the distal tube 502 includes, preferably, a transition section 504 extending longitudinally and coaxially with the distal lumen 503. The transition section 504 defines a transition lumen 505 extending longitudinally along the transition section 504. The transition lumen 505 is defined to provide (provides) a smooth transition (tapered transition) between the proximal lumen 501 and the distal lumen 503. A shoulder section 510 is formed at the end section of the distal tube 502 and the outer portion of the transition section 504. The shoulder section 510 is located or positioned between the proximal tube 500 and the distal tube 502, and the distal tube 502 meets the proximal tube 500 at the shoulder section 510. The shoulder section 510 is configured to contact (abut) the end portion of the proximal tube 500 once the end portion (such as, the transition section 504) of the distal tube 502 is received (at least in part) into the proximal lumen 501 of the proximal tube 500 (as depicted in FIG. 12B). In accordance with an option, the shoulder section 510 is positioned between the proximal tube 500 and distal tube 502. It is the shoulder between the portion of the distal tube 502 received (at least in part) into the proximal lumen 501.The elongated electrically-conductive flexible tube assembly 102 also includes a transition tube 506. In accordance with a preferred option (and not limited thereto), the transition tube 506 includes (defines) a transition lumen 507 configured to receive, at least in part, the distal tube 502. It will be appreciated that, in accordance with another embodiment, transition lumen 505 does not provide a smooth transition (and may be a step transition, if desired). It will be appreciated that, in accordance with another embodiment, that there is no transition section 504 and not transition lumen 505. It will be appreciated that, in accordance with another embodiment, the distal tube 502 has a smaller diameter than the proximal tube 500, thus the distal tube 502 is fit into the proximal tube 500 as a means of joining the tubes together. The distal tube 502 may be fitted (at least in part) into the proximal lumen 501 of the proximal tube 500 with glue (adhesive) such as EPO-TEK (TRADEMARK) Model number 353 ND epoxy (manufactured by EPOXY TECHNOLOGY, INC., based out of the U.S.A.), and/or any equivalent thereof. It will be appreciated that a portion of the distal tube 502 is positioned inside the proximal tube 500 (for adhesion purposes).

Referring to the embodiment (implementation) as depicted in FIG. 12B, the transition section 504 (the portion of the distal tube 502) is not used in the embodiment as depicted in FIG. 12A. In accordance with the embodiment as depicted in FIG. 12A, the transition section 504 is configured to be received (at least in part) into the proximal lumen 501 of the proximal tube 500.

Referring to the embodiment (implementation) as depicted in FIG. 13, the transition tube 506 is installed so that the transition lumen 507 receives, at least in part, the distal tube 502. The transition tube 506 is configured to contact or abut an end section of the proximal tube 500 once the transition tube 506 is installed and moved toward the end section of the proximal tube 500. The transition tube 506 is configured to provide a smooth transition for the outer surface of the proximal tube 500 and the distal tube 502 (once installed as depicted).

Referring to the embodiment (implementation) as depicted in FIG. 14, the elongated electrically-conductive flexible tube assembly 102 also includes an electrical insulation layer 508 formed, at least in part, over the transition tube 506, the distal tube 502 and the proximal tube 500. The electrical insulation layer 508 includes a coating of a Polytetrafluoroethylene (PTFE) heat-shrink insulation, a paralene dielectric coating, and/or any equivalent thereof.

FIG. 15 and FIG. 16 depict side views in accordance with the first embodiment (implementation) of the elongated medical needle assembly 100 of FIG. 1 in combination with the technical feature(s) of FIG. 12A. The elongated electrically-conductive flexible tube assembly 102 of FIG. 15 and FIG. 16 is adapted such that the elongated electrically-conductive flexible tube assembly 102 includes a proximal tube and a distal tube (positioned one after the other), whereas the elongated electrically-conductive flexible tube assembly 102 of FIG. 1 depicts a single tube (or a single tapered tube).

Referring to the embodiment (implementation) as depicted in FIG. 15, the transition tube 506 is not used (deployed or installed). The electrically-exposed outer surface 108 is, preferably, located on the distal tube 502 and is spaced apart from the proximal tube 500 (as depicted in FIG. 15 and/or FIG. 17).

Referring to the embodiment (implementation) as depicted in FIG. 16, the transition tube 506 is installed to the distal tube 502.

FIG. 17 and FIG. 18 depict side views in accordance with the second embodiment (implementation) of the elongated medical needle assembly 100 of FIG. 2 in combination with the technical feature(s) of FIG. 12A. The elongated electrically-conductive flexible tube assembly 102 of FIG. 17 and FIG. 18 includes a proximal tube and a distal tube, whereas the elongated electrically-conductive flexible tube assembly 102 of FIG. 2 includes a single tube (or a single tapered tube).

Referring to the embodiment (implementation) as depicted in FIG. 17, the transition tube 506 is not used (deployed or installed).

Referring to the embodiment (implementation) as depicted in FIG. 18, the transition tube 506 is installed to the distal tube 502.

FIG. 19 and FIG. 20 depict side views in accordance with the third embodiment (implementation) of the elongated medical needle assembly 100 of FIG. 9 in combination with the technical feature(s) of FIG. 12A. The elongated electrically-conductive flexible tube assembly 102 of FIG. 19 and FIG. 20 includes a proximal tube and a distal tube, whereas the elongated electrically-conductive flexible tube assembly 102 of FIG. 9 depicts a single tube (or a single tapered tube).

Referring to the embodiment (implementation) as depicted in FIG. 19, the transition tube 506 is not used (deployed or installed). The electrically-exposed outer surface 108 is, preferably, located on the proximal tube 500 and is spaced apart from the distal tube 502 (as depicted in FIG. 19).

Referring to the embodiment (implementation) as depicted in FIG. 20, the transition tube 506 is installed to the distal tube 502.

FIG. 21, FIG. 22 and FIG. 23 depict, for the purpose of a side-by-side comparison, a side view (FIG. 21) of the first embodiment (implementation) of the elongated medical needle assembly 100 of FIG. 1, a side view (FIG. 22) of the second embodiment (implementation) of the elongated medical needle assembly 100 of FIG. 2, and a side view (FIG. 23) of the third embodiment (implementation) of the elongated medical needle assembly 100 of FIG. 9.

Referring to the embodiment (implementation) as depicted in FIG. 21 (for the first embodiment), FIG. 22 (for the second embodiment) and FIG. 23 (for the third embodiment), there is an emphasis on the geometry for the first radius 301 and the second radius 302.

FIG. 24 depicts a side view of any embodiment (implementation) of the elongated medical needle assembly 100 of FIG. 1, FIG. 2 or FIG. 9.

Referring to the embodiment (implementation) as depicted in FIG. 24, the elongated electrically-conductive flexible tube assembly 102 is configured to be mounted to a handle assembly 400. A cable assembly 402 is configured to extend from the handle assembly 400. A connector assembly 404 is mounted to an end portion of the cable assembly 402. The connector assembly 404 is configured to be electrically connected to an energy generator (known and not depicted, such as a radiofrequency generator, etc.) configured to generate energy (such as, radiofrequency energy). This is done in such a way that the energy (that is generated by the energy generator) may travel along the cable assembly 402, along an electrically-conductive portion (of the elongated electrically-conductive flexible tube assembly 102) and then to the electrically-exposed outer surface 108 (also called the electrode). The lumen portal 107 of the elongated lumen 106 are configured to selectively receive (and/or guide) a medical element from the proximal end to the distal end of the elongated electrically-conductive flexible tube assembly 102. The medical element, for instance, may include a contrast dye material, a guidewire assembly, etc., and any equivalent thereof. The contrast dye material may be injected into the elongated lumen 106 at the proximal end (that is, from the handle assembly 400). The contrast dye material is a medical element configured to be used by (detected by) a medical-imaging system (known and not depicted). The guidewire assembly may be advanced into the elongated lumen 106 at the proximal end (that is, from the handle assembly 400).

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus for use on a biological feature of a patient, the apparatus comprising:
an elongated medical needle assembly, including:
an elongated electrically-conductive flexible tube assembly having a distal portion defining an elongated lumen extending longitudinally along an entire length of the elongated electrically-conductive flexible tube assembly toward the distal portion configured to be maneuvered, at least in part, within the patient and toward the biological feature; and
a first electrically-insulated layer covering, at least in part, an outer surface of the elongated electrically-conductive flexible tube assembly; and
a second electrically-insulated layer covering, at least in part, the distal portion of the elongated electrically-conductive flexible tube assembly.

2. The apparatus of claim 1, further comprising an electrically-exposed outer surface being located proximate to the distal portion, and also located between the first electrically-insulated layer and second electrically-insulated layer.

3. The apparatus of claim 2, wherein the electrically-exposed outer surface being configured to selectively emit energy toward the biological feature of the patient in response to selective movement of the energy along the elongated electrically-conductive flexible tube assembly toward the electrically-exposed outer surface.

4. The apparatus of claim 3, wherein:
the elongated electrically-conductive flexible tube assembly includes:
a proximal tube; and
a distal tube; and
the electrically-exposed outer surface includes a surface roughness that is greater than the surface roughness of a reminder of the proximal tube.

5. The apparatus of claim 3, wherein:
the elongated electrically-conductive flexible tube assembly includes:
a proximal tube; and
a distal tube; and
the electrically-exposed outer surface includes a surface roughness that is greater than the surface roughness of a reminder of the distal tube.

6. The apparatus of claim 3, wherein the elongated electrically-conductive flexible tube assembly includes two tubes having, respectively, a larger diameter proximal shaft section and a smaller diameter distal shaft section, with the electrically-exposed outer surface positioned therebetween.

7. The apparatus of claim 3, wherein the elongated electrically-conductive flexible tube assembly includes two tubes having, respectively, a larger diameter proximal shaft section and a smaller diameter distal shaft section, with the electrically-exposed outer surface positioned on a distal shaft section.

8. The apparatus of claim 3, wherein the elongated electrically-conductive flexible tube assembly includes two tubes having, respectively, a larger diameter proximal shaft section and a smaller diameter distal shaft section, with the electrically-exposed outer surface positioned on a proximal shaft section.

9. The apparatus of claim 3, wherein:
the elongated electrically-conductive flexible tube assembly includes:
a proximal tube; and
a distal tube; and
the electrically-exposed outer surface is located on the proximal tube and is spaced apart from the distal tube.

10. The apparatus of claim 3, wherein:
the elongated electrically-conductive flexible tube assembly includes:
a proximal tube; and
a distal tube; and
the electrically-exposed outer surface is located on the distal tube and is spaced apart from the proximal tube.

11. The apparatus of claim 3, wherein:
the elongated electrically-conductive flexible tube assembly is configured to form a single curve having a single radius;
the electrically-exposed outer surface is located at a distal-most section of the single curve when in the elongated electrically-conductive flexible tube assembly is in its original, curved shape; and
the electrically-exposed outer surface is spaced apart from a portal lumen of the elongated lumen at the distal portion.

12. The apparatus of claim 3, wherein:
the elongated electrically-conductive flexible tube assembly is configured to form:
a first curve having a first radius located at the distal portion; and
a second curve having a second radius, the second curve being spaced apart from the distal portion; and
the electrically-exposed outer surface is located at a distal-most section of a smaller curve when in the elongated electrically-conductive flexible tube assembly is in its original, curved shape, and
the electrically-exposed outer surface is located adjacent to the distal portion and is located in a spaced-apart relationship to a lumen portal.

13. The apparatus of claim 3, wherein:
the elongated electrically-conductive flexible tube assembly includes:
a proximal tube; and
a distal tube; and
an outer diameter of the proximal tube is smaller than the outer diameter of the distal tube; and
the proximal tube defines a proximal lumen extending along a longitudinal axis of the proximal tube; and
the distal tube defines a distal lumen extending along a longitudinal axis of the distal tube; and
the electrically-exposed outer surface is positioned at the proximal tube.

14. The apparatus of claim 3, wherein:
the elongated electrically-conductive flexible tube assembly includes:
a proximal tube; and
a distal tube; and
the proximal tube and the distal tube are adhered together.

15. The apparatus of claim 3, wherein:
the elongated electrically-conductive flexible tube assembly includes:
a proximal tube; and
a distal tube; and
an outer diameter of the proximal tube is smaller than the outer diameter of the distal tube; and
the proximal tube defines a proximal lumen extending along a longitudinal axis of the proximal tube; and
the distal tube defines a distal lumen extending along a longitudinal axis of the distal tube.

16. The apparatus of claim 15, wherein:
the distal tube includes a transition section extending longitudinally and coaxially with the distal lumen; and
the transition section defines a transition lumen extending longitudinally along the transition section; and
the transition lumen is defined to provide a smooth transition between the proximal lumen and the distal lumen.

17. The apparatus of claim 15, wherein:
a shoulder section is formed at an end section of the distal tube and an outer portion of a transition section; and
the shoulder section is configured to contact an end portion of the proximal tube once the end portion of the distal tube is received, at least in part, into the proximal lumen of the proximal tube.

18. The apparatus of claim 15, wherein:
the elongated electrically-conductive flexible tube assembly also includes:
a transition tube including a transition lumen configured to receive, at least in part, the distal tube; and
the transition tube is configured to contact or abut an end section of the proximal tube once the transition tube is installed and moved toward the end section of the proximal tube; and
the transition tube is configured to provide a smooth transition for the outer surface of the proximal tube and the distal tube.

19. An apparatus for use on a biological feature of a patient, the apparatus comprising:
an elongated medical needle assembly, including:
an elongated electrically-conductive flexible tube assembly configured to be maneuvered, at least in part, within the patient and toward the biological feature; and
an electrically-exposed outer surface, being located between spaced-apart electrically-insulated layers, covering, at least in part, an outer surface of the elongated electrically-conductive flexible tube assembly.

20. An apparatus for use on a biological feature of a patient, the apparatus comprising:
an elongated medical needle assembly, including:
an elongated electrically-conductive flexible tube assembly having a distal portion configured to be maneuvered, at least in part, within the patient and toward the biological feature; and
a first electrically-insulated layer covering, at least in part, an outer surface of the elongated electrically-conductive flexible tube assembly; and
a second electrically-insulated layer covering, at least in part, the distal portion of the elongated electrically-conductive flexible tube assembly.
